(19) United States
(12) Patent Application Publication
(10) Pub. No.: US 2006/0233714 A1
(43) Pub. Date: Oct. 19, 2006

(54) Use of a volatile liquid at atmospheric pressure and ambient temperature for the production of pharmaceutical or biological compositions

(76) Inventors: Jean-Louis Assie, Bergerac (FR); Jean-Jacques Huart, Lille (FR)

Correspondence Address: RICHARD M. GOLDBERG, 25 EAST SALEM STREET, SUITE 419, HACKENSACK, NJ, 07601, US

(21) Appl. No.: 10/545,553

(22) PCT Filed: Feb. 13, 2004
(86) PCT No.: PCT/FR04/50055

§ 371 Date: Jun. 15, 2006

(30) Foreign Application Priority Data
Feb. 13, 2003 (FR) 0301761

(51) Int. Cl. A01N 1/02; A61L 9/04

(52) U.S. Cl. 424/45; 435/2

(57) ABSTRACT

A liquid which is a perfluorinated compound whose vapor pressure at 25° C. and atmospheric pressure is greater than or equal to 10,000 Pa (100 mbar) and whose boiling point is greater than or equal to 30° C. is used in the manufacture of a pharmaceutical and/or biological composition. The pharmaceutical and/or biological composition essentially only includes this liquid and the pharmaceutical ingredient in powder form and/or the solid biological material. The pharmaceutical ingredient and/or the biological material are insoluble in the liquid. Optionally, a film-forming agent is also included.

The invention relates to the use of a liquid volatile at atmospheric pressure and room temperature for the manufacture of a pharmaceutical and/or biological composition. It further relates to the pharmaceutical composition comprising such a liquid.

Some pharmaceutical compositions are formulated to be taken orally and consist of a solid, therapeutically active ingredient coated with solid excipients. However, these compositions have a slow therapeutic effect.

It is for this reason that other pharmaceutical compositions are formulated to be injected into the patient's body, in which case the active ingredient is immediately released into the body.

However, these injectable pharmaceutical compositions are not always tolerated by the patient, either because the liquid vehicle in which they are solubilized is itself toxic, or, as in the case of diabetics, because the injections have to be repeated very frequently, which is not always compatible with the patients' lifestyle.

Furthermore, there are also pharmaceutical compositions, e.g. for dermatological use, which are formulated as an ointment, gel or liquid to be applied to the skin.

Here again, however, the excipients in which these compositions are formulated can be toxic or allergizing and can cause adverse reactions.

Pharmaceutical compositions currently exist which can be administered by application to the mucosae, particularly the nasal mucosae. Such formulations contain the active ingredient in liquid form and a propellant gas to bring the active ingredient and its vehicle onto the nasal mucosae.

However, because of the presence of propellant gas, the active ingredient is often brought into the patient's respiratory passages and lungs and this type of composition is only used when such a delivery to the respiratory passages and lungs is necessary.

Other pharmaceutical compositions currently in existence are formulated as a suppository for rectal administration or as a pessary for vaginal administration.

Once again, the active ingredient passes through the anal or vaginal mucosa, respectively.

However, apart from the fact that their mode of administration sometimes makes these compositions poorly accepted by the patient, they have several disadvantages. In particular, on the one hand the excipient melts and causes unpleasant and messy flows, and on the other hand the compositions tend to come out of the anal passage or vagina. This phenomenon is accentuated in the case of suppositories, which induce a defecation reflex, especially in children. The drug is not administered in this case.

Also, some pharmaceutical compositions, particularly those comprising hormones or nicotine, are currently used in the form of a patch to be applied to the skin for release through the skin barrier.

However, particularly in the case of hormones administered to menopausal women, the formulation of the composition to be applied to the patch poses numerous problems because of the instability of the hormones in the current formulations.

A specific case in which the pharmaceutical formulations are unsuitable is that of the care administered to severe bums patients.

In fact, several problems arise in the case of severe bums patients.

First of all, there is the problem of the pain caused by the slightest pressure contact, even when the therapeutic product is applied by means of an atomizer containing a propellant gas.

Then there is the problem of the exact composition of the care product: in fact, some care products have to be administered only to the site of the bum without running over onto healthy areas or areas requiring a different treatment. This is particularly the case of pharmaceutical compositions formulated as an ointment or liquid.

Furthermore, some biological compositions for repairing living tissues consist of stem cells grown in an appropriate culture medium.

The stem cells are harvested and immediately placed in the culture medium in order to grow. It is not known at the present time how to maintain these stem cells at an intermediate growth stage without progressing to the final desired growth, or how to keep them healthy at this intermediate growth stage.

One example, particularly concerning application to severe bums patients, is the growth of human skin in situ: the stem cells are harvested and grown in situ in their culture medium until the culture medium is withdrawn, thus stopping the growth.

It could be advantageous to be able to grow these skin cells in vitro and block the growth of these cells at a certain stage in order to be able to transplant them in situ when the other forms of care administered to severe bums patients have progressed sufficiently to allow such a transplantation.

Also, tests on pharmaceutical compositions and cosmetic compositions for the absence of toxicity and the absence of an allergic type of reaction are increasingly being carried out on artificial skins cultivated in vitro, rather than these compositions being tested on animals or humans.

It would thus be advantageous, for an increased reactivity, to be able to have human skin stem cells which are blocked at a certain growth stage and kept healthy and which could then be grown as far as the formation of skin tissue for the purpose of testing the compositions on fresh human skin tissues, as explained above.

The invention aims to solve the problems of the pharmaceutical or biological compositions of the prior art by proposing compositions which are in a biocompatible carrier liquid that is non-toxic to humans and cytostatic and has a high volatility at room temperature and atmospheric pressure.

The invention thus proposes the use of a liquid which is a perfluorinated compound whose vapor pressure at 25° C. and atmospheric pressure is greater than or equal to 10,000 Pa (100 mbar) and whose boiling point is greater than or equal to 30° C. in the manufacture of a pharmaceutical and/or biological composition consisting only of this liquid and the pharmaceutical ingredient in powder form and/or the solid biological material, the pharmaceutical ingredient and/or the biological material being insoluble in the liquid, and optionally a film-forming agent.

Preferably, the liquid at a vapor pressure at 25° C. and atmospheric pressure of between 10,000 Pa (100 mbar) and 70,000 Pa (700 mbar).

Particularly preferably, the liquid is a hydrofluoro ether (HFE), a mixture of the isomers of a hydrofluoro ether and a mixture of hydrofluoro ethers (HFE) of formula (1) below:
R_{H}—O—R_{F} (1)
in which:
R_{H} is a substituted or unsubstituted, linear, branched or cyclic, partially or totally hydrogenated alkyl radical, and
R_{F} is a linear, branched or cyclic, totally or partially fluorinated alkyl radical.

Particularly preferably, the liquid is selected from the group comprising methoxyheptafluoropropane, ethoxynonafluorobutane and methoxynonafluoro-butane.

In a first variant, the liquid constitutes the cytostatic preserving medium for stem cells of human, animal or vegetable tissues.

In this case the stem cells are preferably stem cells of human skin tissues.

In a second variant, the liquid is the carrier liquid of a pharmaceutical composition in which the active ingredient is in the form of a micronized powder insoluble in the carrier liquid.

A second subject of the invention is a pharmaceutical composition of the type comprising an active ingredient and a carrier liquid, said composition consisting of a carrier liquid which is a perfluorinated compound whose vapor pressure at 25° C. and atmospheric pressure is greater than or equal to 10,000 Pa (100 mbar) and whose boiling point is greater than or equal to 30° C., and a therapeutically active ingredient in the form of a micronized powder insoluble in the carrier liquid, and optionally a film-forming agent.

Preferably, the carrier liquid has a vapor pressure at 25° C. and atmospheric pressure of between 10,000 Pa (100 mbar) and 70,000 Pa (700 mbar).

Particularly preferably, the carrier liquid is a hydrofluoro ether, a mixture of the isomers of a hydrofluoro ether and a mixture of hydrofluoro ethers of formula 1 below:
R_{H}—O—R_{F} (1)
in which:
R_{H} is a substituted or unsubstituted, linear, branched or cyclic, partially or totally hydrogenated alkyl radical, and
R_{F} is a linear, branched or cyclic, totally or partially fluorinated alkyl radical.

The radical R_{F} in formula 1 can have several positional isomers.

For example, the perfluorobutyl radical has two positional isomers, namely perfluoro-n-butyl, CF₃CF₂CF₂CF₂, and perfluoro-n-isobutyl, (CF₃)₂CFCF₂.

Thus, above and below, the term “hydrofluoro ether” encompasses both a hydrofluoro ether whose radicals R_{H} and R_{F} are each independently present in a single isomeric form and a hydrofluoro ether whose radical R_{H} and/or radical R_{H} are each independently present in the form of a mixture of two or more of their isomeric forms.

Likewise, above and below, the term “mixture of hydrofluoro ethers” encompasses mixtures of two or more hydrofluoro ethers having different radicals R_{H} and R_{F}, each radical R_{H} and R_{F} independently being in a single isomeric form or in the form of a mixture of several of their isomeric forms.

Particularly preferably, the carrier liquid is selected from the group comprising methoxyheptafluoropropane, ethoxynonafluorobutane and methoxy-nonafluorobutane.

As far as the active ingredient is concerned, this is selected particularly from insulin, vaccines, analgesics, Trinitrine®, nicotine, iodine, hormones, antibiotics and antibacterials.

In one preferred embodiment of the pharmaceutical composition of the invention, the volume ratio of active ingredient to carrier liquid is between 1/4 and 4/1 inclusive.

Particularly preferably, the volume ratio of active ingredient to carrier liquid is 1/1.

In one particular mode of carrying out the invention, the pharmaceutical composition is packaged in an atomizer.

Said composition preferably contains a film-forming agent.

A third subject of the invention is the use of the pharmaceutical composition of the invention for the care of severe burns patients.

The invention will be understood more clearly and other characteristics and advantages thereof will become more clearly apparent from the explanatory description which follows.

In a first embodiment, the invention relates to pharmaceutical compositions which have to be delivered rapidly into the blood, lymphatic or other system of the body, this generally being effected by injection with a syringe.

Now, apart from the fact that the use of syringes is generally poorly accepted by patients and poses certain health safety problems due to the transmission of various diseases, some active ingredients are only soluble in toxic solvents like castor oil.

In this case the invention proposes maintaining the active ingredient in solid form or converting it to a solid form, for example by lyophilization, and applying it to the mucosae.

The active ingredient in powder form must be a micronized powder and must be deposited on the mucosae, particularly nasal mucosae.

In the prior art this is generally effected by packaging the active ingredient in a vaporizer, more particularly a nasal vaporizer.

The active ingredient in micronized powder form is propelled by a propellant gas under pressure, which limits its use to active ingredients that can and/or have to be transmitted to the respiratory passages and lungs, thereby limiting its use.

To solve this problem, in the invention the active ingredient will not be propelled by a propellant gas, but will simply be deposited at the precise site of use, i.e. the mucosae, without being propelled into the respiratory passages and lungs, through the use of a carrier in liquid form that is very volatile at room temperature and atmospheric pressure.

The vapor pressure at 25° C. and atmospheric pressure of such a carrier liquid is greater than or equal to 10,000 Pa (100 mbar) and preferably between 10,000 Pa (100 mbar) and 70,000 Pa (700 mbar), and its boiling point must be greater than or equal to 30° C.

In fact, if it had a boiling point below 30° C., the carrier liquid would no longer be in liquid form at room temperature, but in gaseous form or in the form of a liquid-gas mixture, giving rise to the same problem as that associated with the use of a propellant gas.

A carrier liquid having a vapor pressure at 25° C. and atmospheric pressure of 10,000 Pa (100 mbar) volatilizes in about 10 seconds at atmospheric pressure and room temperature, which is compatible with the desired use on the mucosae.

The higher the vapor pressure, the greater is the volatility of the carrier liquid. With a carrier liquid whose vapor pressure at 25° C. and atmospheric pressure is greater than 70,000 Pa (700 mbar), the evaporation is of the flash type and produces a cooling of the substrate to which it is applied.

If this substrate is a human or animal mucosa, such a sudden cooling may cause unwanted phenomena such as vasoconstriction or discomfort during administration.

It is for this reason that, in the invention, it is preferred to use a carrier liquid whose vapor pressure at 25° C. and atmospheric pressure is definitely greater than 10,000 Pa (100 mbar), but also less than or equal to 70,000 Pa (700 mbar).

One particularly preferred carrier liquid within the framework of the present invention is a hydrofluoro ether or a mixture of hydrofluoro ethers of formula (1):
R_{H}—O—R_{F} (1)
in which:
R_{H} is a substituted or unsubstituted, linear, branched or cyclic, partially or totally hydrogenated alkyl radical, and
R_{F} is a linear, branched or cyclic, totally or partially fluorinated alkyl radical.

In fact, hydrofluoro ethers are highly volatile, biocompatible compounds that are devoid of toxicity to humans and chemically inert towards virtually all organic products.

The HFE used in the invention are intended to be brought into contact particularly with human tissues.

It is therefore important for their toxicity and their ability to dissolve human tissues to be as low as possible.

For this reason, R_{H} will preferably be an alkyl radical with a short carbon chain. Particularly preferably, the radical R_{H} will be an alkyl radical having 1 or 2 carbon atoms.

As regards the necessary volatility of the HFE constituting the carrier liquid of the composition of the invention, this is obtained by varying the chain length of the radical R_{F}, since this is how it is possible to vary the vapor pressure of the HFE.

For an HFE having a vapor pressure at 25° C. and atmospheric pressure of 28,000 Pa (280 mbar), the evaporation time when 1 ml is brought into contact with living human hands was about 4 seconds; for an HFE having a vapor pressure at 25° C. and atmospheric pressure of 14,000 Pa (140 mbar), the evaporation time was 7 seconds; for an HFE having a vapor pressure at 25° C. and atmospheric pressure of 2100 Pa (21 mbar), the evaporation time was about 15 seconds; and for an HFE having a vapor pressure at 25° C. and atmospheric pressure of 69,800 Pa, the evaporation time was about 1 s.

It is for this reason that, within the framework of the invention, the carrier liquid used in the composition of the invention will particularly preferably be methoxyheptafluoropropane having a vapor pressure at 25° C. and atmospheric pressure of 69,800 Pa (698 mbar) (evaporation time of about 1 s on the hands), marketed by 3M under the reference HFE 7000, methoxynonafluorobutane having a vapor pressure at 25° C. and atmospheric pressure of 28,000 Pa (280 mbar) (evaporation time of about 4 seconds on the hands), marketed by 3M under the reference HFE 7100, or ethoxynonafluorobutane having a vapor pressure at 25° C. and atmospheric pressure of 14,000 Pa (140 mbar) (evaporation time of about 7 seconds on the hands), marketed by 3M under the reference HFE 7200.

The evaporation times indicated above are averages for 2 ml applied to the skin. They depend in particular on the temperature of the substrate to which they are applied and can vary between individuals, for application to the skin and mucosae, according to the body temperature and room temperature.

Using such a carrier liquid avoids damaging the mucosae by the repeated use of a propellant gas, which applies a certain pressure to the mucosae.

Furthermore, a pharmaceutical composition as formulated in the invention is particularly appropriate for depositing the desired active ingredients on deep wounds, such as wounds on severe burns patients, which cannot easily withstand the simple manual pressure needed to apply a compress.

Also, the active ingredient is delivered only to the desired site and, being in powder form, does not flow and does not run over onto the tissues surrounding the wound.

Thus one particular application of the pharmaceutical composition of the invention is for the care of severe bums patients.

In all cases the active ingredient contained in the pharmaceutical composition of the invention will be any active ingredient that can be converted to a micronized powder, either by lyophilization, or because it is already synthesized in solid form, or because it has been converted to this powder form, for example by lyophilization.

Thus the active ingredient can replace vaccines currently administered by injection into the body, insulin which has to be released immediately into the body, analgesics for severe pain, antibiotics and antiasthmatics, as well as all heart regulators such as Trinitrine®.

All these products that are normally injected or vaporized by means of a propellant gas will be able to be administered to the mucosae using the pharmaceutical composition of the invention.

Likewise, the dermatological compositions that are currently formulated with excipients, particularly dermatological compositions containing antibacterials, will be able to be formulated by virtue of the invention without any allergizing excipient.

Similarly, the active ingredients that are currently formulated for oral administration will be able to be formulated according to the invention in the form of a micronized powder in the carrier liquid of the invention.

It will be possible for these active ingredients to be analgesics or iodine cachets.

One particular application relates to nicotine, which will be able to have an immediate effect when administered in this way.

However, it will be possible to administer nicotine both to the mucosae and to the skin, a film being formed.

In this case the composition of the invention will advantageously also contain a film-forming agent, making it possible to keep the nicotine in place on the skin by forming a protective film over the nicotine.

In the same way, the hormones that are currently administered in patch form, such as hormones for treating the menopause, will be able to be administered in the form of the composition of the invention, preferably containing a film-forming agent.

Hormones are actually very difficult to formulate in patch form since they are unstable in the current formulations.

In the pharmaceutical composition of the invention, the carrier liquid acts as a vehicle for transport to the application site, so it will be possible to formulate the composition of the invention in the form of a single-dose or multidose atomizer.

Numerous atomizers for administering precise doses, either once or several times in succession, are known in the state of the art.

Preferably, for a good efficacy and a good transport capacity, the composition of the invention will consist of 1-4 volumes of active ingredient in micronized powder form to 4-1 volumes of carrier liquid.

Very particularly preferably, the formulations of the pharmaceutical composition of the invention will consist of one volume of active ingredient in micronized powder form and one volume of carrier liquid.

Virtually all organic compounds are insoluble in liquid HFE, guaranteeing the chemical integrity of the active ingredient in the HFE and hence the total preservation of the therapeutic properties of said active ingredient.

Furthermore, HFE exhibit no toxicity to humans and are non-toxic to the ozone layer and the environment.

In a second embodiment, the invention relates to biological compositions in which the biological product is in solid form and is insoluble in a carrier liquid that is identical to the carrier liquid described for the pharmaceutical composition according to the first embodiment of the invention.

Thus the invention is based on the use of a carrier liquid as described above for the manufacture of a pharmaceutical and/or biological composition.

One particularly preferred biological composition in the second embodiment of the invention consists of stem cells of human, animal or vegetable tissues.

In fact, in particular, the hydrofluoro ethers of formula 1 above are cytostatic, which means that any stem cell or living cell will be perfectly preserved and maintained at a certain growth stage without being damaged.

It would thus be possible to prepare in advance stem cells of living tissue, such as human tissues, animal tissues or vegetable tissues, at a certain stage of their growth, and then continue the growth to the desired stage by opening the container in which they are conditioned in hydrofluoro ethers and, as the hydrofluoro ether or mixture of hydrofluoro ethers will be immediately volatilized, replacing it with a culture medium in order to continue the growth of these cells.

It will be possible to use these stem cells to reconstitute skin on severe burns patients or very rapidly to produce samples of fresh human skin for the in vitro testing of cosmetic and/or pharmaceutical products for non-toxicity.

As the carrier liquid is volatile, it will be clearly apparent to those skilled in the art that the pharmaceutical and/or biological composition of the invention is to be placed in a closed container that can be opened and optionally reclosed and is airtight.

Of course, the invention is in no way limited to the embodiments described and illustrated, which have been given only as examples.

On the contrary, the invention comprises all the technical equivalents described, as well as their combinations if these are effected within the spirit of the invention.

1. Use of a liquid which is a perfluorinated compound whose vapor pressure at 25° C. and atmospheric pressure is greater than or equal to 10,000 Pa (100 mbar) and whose boiling point is greater than or equal to 30° C. in the manufacture of a pharmaceutical and/or biological composition consisting essentially of:
said liquid, and
at least one of:
a pharmaceutical ingredient in powder form, and
a solid biological material,
the at least one of the pharmaceutical ingredient the biological material being insoluble in a carrier liquid.

2. Use according to claim 1, wherein the liquid has a vapor pressure at 25° C. and atmospheric pressure of between 10,000 Pa (100 mbar) and 70,000 Pa (700 mbar).

3. Use according to claim 1, wherein the carrier liquid is selected from the group consisting of a hydrofluoro ether (HFE), a mixture of the isomers of a hydrofluoro ether and a mixture of hydrofluoro ethers (HFE) of formula (1) below:
R_{H}—O—R_{F} (1)
in which:
R_{H} is a substituted or unsubstituted, linear, branched or cyclic, partially or totally hydrogenated alkyl radical, and
R_{F} is a linear, branched or cyclic, totally or partially fluorinated alkyl radical.

4. Use according to claim 1, wherein the carrier liquid is selected from the group consisting of methoxyheptafluoropropane, ethoxynonafluorobutane and methoxynonafluorobutane.

5. Use according to claim 1, wherein the carrier liquid constitutes a cytostatic preserving medium for stem cells of human, animal or vegetable tissues.

6. Use according to claim 5, wherein the stem cells are stem cells of human skin tissues.

7. Use according to claim 1, wherein the liquid is the carrier liquid of a pharmaceutical composition having an active ingredient in the form of a micronized powder insoluble in the carrier liquid.

8. Pharmaceutical composition of the type comprising an active ingredient and a carrier liquid, said composition consisting essentially of:
a carrier liquid which is a perfluorinated compound whose vapor pressure at 25° C. and atmospheric pressure is greater than or equal to 10,000 Pa (100 mbar) and whose boiling point is greater than or equal to 30° C., and
a therapeutically active ingredient in the form of a micronized powder insoluble in the carrier liquid.

9. Pharmaceutical composition according to claim 8, wherein the carrier liquid has a vapor pressure at 25° C. and atmospheric pressure of between 10,000 Pa (100 mbar) and 70,000 Pa (700 mbar).

10. Pharmaceutical composition according to claim 8, wherein, the carrier liquid is a hydrofluoro ether or a mixture of hydrofluoro ethers of formula 1 below:
R_{H}—O—R_{F} (1)
in which:
R_{H} is a substituted or unsubstituted, linear, branched or cyclic, partially or totally hydrogenated alkyl radical, and
R_{F} is a linear, branched or cyclic, totally or partially fluorinated alkyl radical.

11. Pharmaceutical composition according to claim 8, wherein the carrier liquid is selected from the group consisting of methoxyheptafluoropropane, ethoxynonafluorobutane and methoxynonafluorobutane.

12. Pharmaceutical composition according to claim 8, wherein the pharmaceutical composition has an active ingredient selected from the group consisting of insulin, vaccines, analgesics, Trinitrine®, nicotine, iodine, hormones, antibiotics and antibacterials.

13. Pharmaceutical composition according to claim 8, wherein a volume ratio of active ingredient to carrier liquid is between 1/4 and 4/1 inclusive.

14. Pharmaceutical composition according to claim 13, wherein the volume ratio of active ingredient to carrier liquid is 1/1.

15. Pharmaceutical composition according to claim 8, wherein the pharmaceutical composition is packaged in an atomizer.

16. Pharmaceutical composition according to aim 8, wherein the pharmaceutical composition contains a film-forming agent.

17. Use of the pharmaceutical composition according to claim 8, for the care of severe burn patients.

18. Use of the pharmaceutical composition according to claim 1, further including a film forming agent.
